(19) United States
(12) Patent Application Publication
(10) Pub. No.: US 2023/0235001 A1
(43) Pub. Date: Jul. 27, 2023

(54) NOVEL GAIN-OF-FUNCTION MUTANT OF BMPR2 GENE AND USE THEREOF

(71) Applicants: SOOKMYUNG WOMEN'S UNIVERSITY INDUSTRY-ACADEMIC COOPERATION FOUNDATION, Seoul (KR); SEOUL NATIONAL UNIVERSITY R&DB FOUNDATION, Seoul (KR)

(72) Inventors: Yonghwan KIM, Seoul (KR); Myung-Jin KIM, Seoul (KR); Tae Joon CHO, Seoul (KR)

(73) Assignees: SOOKMYUNG WOMEN'S UNIVERSITY INDUSTRY-ACADEMIC COOPERATION FOUNDATION, Seoul (KR); SEOUL NATIONAL UNIVERSITY R&DB FOUNDATION, Seoul (KR)

(21) Appl. No.: 18/011,365

(22) PCT Filed: Dec. 29, 2020
(86) PCT No.: PCT/KR2020/019261

§ 371 Date: Dec. 19, 2022

(30) Foreign Application Priority Data
Jun. 19, 2020 (KR) 10-2020-0075323

(51) Int. Cl. C07K 14/51; A61P 19/00

(52) U.S. Cl. CPC C07K 14/51; A61P 19/00

(57) ABSTRACT

Disclosed is a technique for identifying a mutation of a particular gene as a new case of a FOP-like phenotype, in addition to the existing ACVR1-R206H mutation known as a cause of FOP and utilizing the identified mutation in the bone disease treatment through osteogenic differentiation. There is provided a bone morphogenetic protein type 2 receptor (BMPR2)-E376K mutant in which the 376th amino acid glutamic acid (E) is mutated into lysine (K) in the BMPR2 gene encoding BMPR2.

### ACKNOWLEDGEMENT

The present invention was made with the support of the Korean Ministry of Science, ICT & Future Planning in 2020, under Project No. 1711043428, which was conducted in the research project “Identification of Pathogenesis through the Study of Biological Functions of Genes Causing Rare and Intractable Skeletal Disorders” within the project “Genome Future Source Technology Development Pilot Project,” with the support of the Korean Ministry of Science and ICT in 2020, under Project No. 1711112814, which was conducted in the research project “Research on the Function of TONSL Complexes in the Process of DNA Damage Repair and DNA Replication” within the project “(Type 1-2) Mid-level Research (within an average annual research cost of 100 to 200 million won),” and with the support of the Korean Ministry of Science and ICT in 2020, under Project No. 1711105488, which was conducted in the research project “Cellular Heterogeneity Research Center” within the project “Science and Engineering Technology Sector (S/ERC).”

### CROSS-REFERENCE TO RELATED APPLICATIONS

This application is a National Stage Patent Application of PCT International Patent Application No. PCT/KR2020/019261 (filed on Dec. 29, 2020) under 35 U.S.C. § 371, which claims priority to Korean Patent Application No. 10-2020-0075323 (filed on Jun. 19, 2020), which are all hereby incorporated by reference in their entirety.

### SEQUENCE LISTING

This application contains a Sequence Listing submitted via EFS-Web and hereby incorporated by reference in its entirety. The Sequence Listing is named “FP22-09046_US_ST25.txt”, created on Dec. 19, 2022, and 11,806 bytes in size.

### BACKGROUND

The present invention relates to a novel mutant and a use thereof, and more specifically, to a gain-of-function mutant in the BMPR2 gene derived from a patient with Fibrodysplasia ossificans progressiva (FOP), and a use thereof.

Fibrodysplasia ossificans progressiva (FOP, MIM #135100) is a rare autosomal hereditary skeletal disorder characterized by progressive heterotopic bone formation in connective tissues, resulting in disabling conditions (see non-patent documents 1 and 2). A typical feature of patients presenting with FOP is the congenital malformation of a big toe (see non-patent documents 3). About 70% of FOP patients experience episodic pre-osseous inflammatory soft tissue swellings, known as flare-ups, which precede heterotopic ossification (see non-patent documents 4).

Understanding the molecular basis of FOP has been spurred by the identification of causative gain-of-function mutations of the gene encoding ACVR1, a type 1 TGF-beta receptor responsible for bone morphogenetic protein (BMP) signaling (see non-patent document 5). To date, a number of ACVR1 variants have been identified, although 95% of FOP cases are attributed to the ACVR1-R206H mutation (see non-patent documents 6 and 7).

Accumulated evidence supports the idea that the gain-of-function mutation in ACVR1 constitutively stimulates BMP signaling even in the absence of BMP ligands, leading to heterotopic ossifications (see non-patent documents 8 to 10). Interestingly, it was demonstrated that activin A, a cytokine antagonizing BMP signaling in a wild-type (WT) ACVR1 background, specifically enhances BMP signaling within cells harboring the ACVR1-R206H mutation (see non-patent documents 11 and 12). The involvement of activin A in FOP has been validated in the FOP mouse model by showing that treatment with an antibody against activin A reduces heterotopic bone formation (see non-patent document 11). The exact molecular basis of how activin A forces ACVR1 mutation-dependent hyperactivation of BMP signaling remains elusive. In addition, the limited amount of information about the genetic causes of FOP hampers our understanding of the pathophysiology of the disease.

### Non-Patent Document

1. Shore E M, Kaplan F S. Inherited human diseases of heterotopic bone formation. Nat Rev Rheumatol 2010; 6:518-27.

2. Kaplan F S, Le Merrer M, Glaser D L, et al. Fibrodysplasia ossificans progressiva. Best Pract Res Clin Rheumatol 2008; 22:191-205.

3. Towler O W, Shore E M, Xu M, et al. The congenital great toe malformation of fibrodysplasia ossificans progressiva?—A close call. Eur J Med Genet 2017; 60:399-402.

4. Pignolo R J, Bedford-Gay C, Liljesthrom M, et al. The Natural History of Flare-Ups in Fibrodysplasia Ossificans Progressiva (FOP): A Comprehensive Global Assessment. J Bone Miner Res 2016; 31:650-6.

5. Shore E M, Xu M, Feldman G J, et al. A recurrent mutation in the BMP type I receptor ACVR1 causes inherited and sporadic fibrodysplasia ossificans progressiva. Nat Genet 2006; 38:525-7.

6. Pignolo R J, Baujat G, Brown M A, et al. Natural history of fibrodysplasia ossificans progressiva: cross-sectional analysis of annotated baseline phenotypes. Orphanet J Rare Dis 2019; 14:98.

7. Haupt J, Xu M, Shore E M. Variable signaling activity by FOP ACVR1 mutations. Bone 2018; 109:232-40.

8. Yu P B, Deng D Y, Lai C S, et al. BMP type I receptor inhibition reduces heterotopic [corrected] ossification. Nat Med 2008; 14:1363-9.

9. Chakkalakal S A, Zhang D, Culbert A L, et al. An Acvrl R206H knock-in mouse has fibrodysplasia ossificans progressiva. J Bone Miner Res 2012; 27:1746-56.

10. Culbert A L, Chakkalakal S A, Theosmy E G, Brennan T A, Kaplan F S, Shore E M. Alk2 regulates early chondrogenic fate in fibrodysplasia ossificans progressiva heterotopic endochondral ossification. Stem Cells 2014; 32:1289-300.

11. Hatsell S J, Idone V, Wolken D M, et al. ACVR1R206H receptor mutation causes fibrodysplasia ossificans progressiva by imparting responsiveness to activin A. Sci Transl Med 2015; 7:303ra137.

12. Hino K, Ikeya M, Horigome K, et al. Neofunction of ACVR1 in fibrodysplasia ossificans progressiva. Proc Natl Acad Sci USA 2015; 112:15438-43.

13. Machado R D, Southgate L, Eichstaedt C A, et al. Pulmonary Arterial Hypertension: A Current Perspective on Established and Emerging Molecular Genetic Defects. Hum Mutat 2015; 36:1113-27.

14. Wang Y, Wu M H, Cheung M P L, et al. Reprogramming of Dermal Fibroblasts into Osteo-Chondrogenic Cells with Elevated Osteogenic Potency by Defined Transcription Factors. Stem Cell Reports 2017; 8:1587-99.

15. Wright V, Peng H, Usas A, et al. BMP4-expressing muscle-derived stem cells differentiate into osteogenic lineage and improve bone healing in immunocompetent mice. Mol Ther 2002; 6:169-78.

16. Yamamoto K, Kishida T, Sato Y, et al. Direct conversion of human fibroblasts into functional osteoblasts by defined factors. Proc Natl Acad Sci USA 2015; 112:6152-7.

17. Takacs R, Matta C, Somogyi C, Juhasz T, Zakany R. Comparative analysis of osteogenic/chondrogenic differentiation potential in primary limb bud-derived and C3H10T1/2 cell line-based mouse micromass cultures. Int J Mol Sci 2013; 14:16141-67.

18. Pauklin S, Vallier L. Activin/Nodal signalling in stem cells. Development 2015; 142:607-19.

19. Namwanje M, Brown C W. Activins and Inhibins: Roles in Development, Physiology, and Disease. Cold Spring Harb Perspect Biol 2016; 8.

20. Anderson G J, Darshan D. Small-molecule dissection of BMP signaling. Nat Chem Biol 2008; 4:15-6.

21. Zhang L, Zhou F, ten Dijke P. Signaling interplay between transforming growth factor-beta receptor and PI3K/AKT pathways in cancer. Trends Biochem Sci 2013; 38:612-20.

22. Southgate L, Machado R D, Graf S, Morrell N W. Molecular genetic framework underlying pulmonary arterial hypertension. Nat Rev Cardiol 2019.

23. Kim M J, Park S Y, Chang H R, et al. Clinical significance linked to functional defects in bone morphogenetic protein type 2 receptor, BMPR2. BMB Rep 2017; 50:308-17.

24. Hopper R K, Moonen J R, Diebold I, et al. In Pulmonary Arterial Hypertension, Reduced BMPR2 Promotes Endothelial-to-Mesenchymal Transition via HMGA1 and Its Target Slug. Circulation 2016; 133:1783-94.

25. Wang X, Li F, Xie L, et al. Inhibition of overactive TGF-beta attenuates progression of heterotopic ossification in mice. Nat Commun 2018; 9:551.

26. Chang H R, Cho S Y, Lee J H, et al. Hypomorphic Mutations in TONSL Cause SPONASTRIME Dysplasia. Am J Hum Genet 2019; 104:439-53.

27. Li H, Durbin R. Fast and accurate short read alignment with Burrows-Wheeler transform. Bioinformatics 2009; 25:1754-60.

28. Li H, Handsaker B, Wysoker A, et al. The Sequence Alignment/Map format and SAMtools. Bioinformatics 2009; 25:2078-9.

29. McKenna A, Hanna M, Banks E, et al. The Genome Analysis Toolkit: a MapReduce framework for analyzing next-generation DNA sequencing data. Genome Res 2010; 20:1297-303.

30. Wang K, Li M, Hakonarson H. ANNOVAR: functional annotation of genetic variants from high-throughput sequencing data. Nucleic Acids Res 2010; 38:e164.

31. Richards S, Aziz N, Bale S, et al. Standards and guidelines for the interpretation of sequence variants: a joint consensus recommendation of the American College of Medical Genetics and Genomics and the Association for Molecular Pathology. Genet Med 2015; 17:405-24.

### SUMMARY

The present invention relates to a novel case of the FOP-like phenotype in addition to the existing ACVR1-R206H mutation, which is known to cause FOP, and is to present a technology in which a mutation of a specific gene is found and can be utilized to treat bone disease through osteogenic differentiation.

In a first aspect for solving the problem above, the present invention provides a BMPR2-E376K mutant in which an amino acid 376 of a bone morphogenetic protein type 2 receptor (BMPR2) gene encoding BMPR2 is mutated from glutamic acid (E) to lysine (K).

In addition, there is provided the BMPR2-E376K mutant characterized by having a point mutation of guanine (G) to adenine (A) at nucleotide 1126.

In addition, there is provided the BMPR2-E376K mutant characterized by causing a phenotype of Fibrodysplasia ossificans progressiva (FOP).

In addition, there is provided the BMPR2-E376K mutant characterized by being used to treat bone disease through osteogenic differentiation.

In a second aspect for solving the problem above, the present invention provides a cell line including the mutant.

The present invention relates to a novel case of the FOP-like phenotype. The patient developed flare-ups and heterotopic bone formation throughout the body, which is similar to the phenotype of the ACVR1 mutation. However, the proband does not have big toe anomaly or any mutation in the ACVR1 gene. From whole exome sequencing, the present inventors identified and functionally validated a causative gain-of-function mutation in the bone morphogenetic protein type 2 receptor (BMPR2) gene, which encodes a type II TGF-beta receptor in the BMP signaling cascade. This is the first report of a BMPR2 gain-of-function mutation and the resultant FOP-like phenotype. Identification of additional genetic alterations leading to the FOP-like phenotype will be informative regarding the molecular basis of the disease and the biology of TGF/BMP signaling in general.

### BRIEF DESCRIPTION OF THE DRAWINGS

FIGS. 1a to 1h are photos and graphs related to the Identification of a novel variant in a patient with FOP.

FIG. 1a shows radiographs of the proband at age 16 years. Arrows denote the ectopic bone formation in the skeletal muscle. Unlike the typical FOP patient, he has no big toe anomaly.

FIG. 1B is the pedigree chart of the BMPR2 variant in the family. The arrow indicates the proband.

FIG. 1c shows whole exome sequencing detected a heterozygous de novo missense mutation in the BMPR2 gene. The proband has a point mutation of guanine (G) to adenine (A) at nucleotide 1126.

FIG. 1d is schematic of functional domains and the site of mutation in the BMPR2 protein. The mutation is within exon 8 in the kinase domain (KD) of BMPR2. TM, transmembrane; ECD, extracellular domain; CTD, C-terminal domain.

FIG. 1e shows the interspecies homology of the kinase domain of BMPR2. The conserved sequence is indicated by gray shading and the E376K variant is red.

In FIG. 1f, expression of SMAD signaling (p-SMAD1/5/9, SMAD5, p-SMAD2, SMAD2), its downstream target genes (ID1, ID3), and osteogenic differentiation markers (osteocalcin, ALP, RUNX2) in normal (BJ fibroblast) and FOP patient cells were examined by western blotting. Normal and patient cells were cultured under complete media (15% serum) or low serum media (2% horse serum) for inducing osteogenic differentiation.

FIG. 1g shows alkaline phosphatase (ALP) staining (left panel) of the normal and patient fibroblasts after differentiation induced by BMP2 or BMP4 (50 ng/ml) for 2 days. ALP is detected as purple color. ALP activity (right panel) was evaluated by measuring absorbance at 405 nm and total protein was measured using a Micro-BCA protein assay kit and read at 560 nm. The ALP activity was normalized to the protein content of the samples.

FIG. 1h shows Alizarin Red S staining indicative of calcium deposits in cells.

FIGS. 2a to 2g are photos and graphs related to the functional validation of pathogenicity of the BMPR2-E376K variant.

FIG. 2a is schematic of the human BMPR2 gene, showing the G1126A mutation in exon 8 (asterisk and underline). The G-to-A change leads to glutamic acid (upper line)-to-lysine (underline) mutation in the protein sequence. In clones corrected by CRISPR-Cas9, knock-out #3 had a 1 base pair (bp) deletion (hyphen) at nucleotide 1122, with the alleles in a 1:1 ratio. This resulted in a STOP signal. Knock-out #5 had a 1 bp insertion of nucleotide A (double underline), causing a STOP codon mutation. Finally, knock-in #203 contained a normal nucleotide guanine at 1126 and a CTC sequence from the HDR donor template, which preserved the WT amino acid sequence.

FIG. 2b shows protein expression determined by immunoblotting the lysates of the knock-out (#3 and #5) and knock-in (#203) clones edited by CRISPR-Cas9 in patient-derived dermal fibroblasts.

FIG. 2c shows western blot analysis to validate change of BMP signaling by BMPR2-E376K variant in HEK293T cells. HEK293T cells were transiently transfected with constructs expressing empty vector (EV), wild-type (WT), and mutant (mut) versions of BMPR2 (E376K) and ACVR1 (R206H) and then protein expression in the cell lysates was detected by immunoblotting.

FIG. 2d shows the promoter reporter assay in which luciferase expression is regulated by BMP-responsive elements in HEK293T cells transfected with plasmids carrying WT and mutant (mut) BMPR2 and ACVR1.

In FIG. 2e, C3H10T1/2 cells were transfected with empty vector (EV) or HA-tagged WT or mutant BMPR2 and their lysates examined for enhanced phosphorylation of SMAD1/5/9 and expression of downstream effectors ID1, ID3, and SOX9 by western blot analysis. The asterisk indicates a cross-reactive artifact.

FIG. 2f shows Alcian blue staining to detect chondrogenic capacity in C3H10T1/2 cells expressing BMPR2-WT or BMPR2-E376K under BMP2 treatment for 3 weeks. Quantification of staining was determined by measuring absorption at 600 nm.

FIG. 2g shows the mRNA expression of Col2a1, Aggrecan, and Col10a1 obtained from C3H10T1/2 cells expressing empty vector (EV), BMPR2-WT, or BMPR2-E376K, as analyzed by real-time PCR.

FIGS. 3a to 3g are photos related to the molecular basis of the dominant-negative functions of BMPR2-E376K.

FIG. 3a shows the co-immunoprecipitation of HA-tagged BMPR2-E376K with wild-type (WT) V5-tagged ACVR1.

FIG. 3b shows the additive effect of both ACVR1-R206H and BMPR2-E376K in enhancing BMP signaling, as assessed by western blotting for p-SMAD1/5/9, p-SMAD2, ID1, and ID3.

FIG. 3c shows that phosphorylation of SMAD1/5/9 and SMAD2 increased upon treatment with activin A (50 ng/ml) in HEK293T cells expressing empty vector (EV) or V5-tagged WT or mutant (mut) BMPR2.

FIG. 3d shows the western blot analysis of phosphorylation of SMAD2 in HEK293T cells expressing V5-tagged WT or mutant (mut) ACVR1 or BMPR2.

FIG. 3e shows expression and phosphorylation of SMAD in the presence of siRNA against type I receptors, ALK2 or ALK5, in patient-derived dermal fibroblasts.

FIGS. 3f and 3g show ALP staining of C2C12 cell lines stably overexpressing HA-tagged WT or mutant (mut) BMPR2, with and without treatment with BMP4, dorsomorphin (DM), or 5B431542 (SB).

FIGS. 4a to 4c are Scheme of BMPR2 genomic DNA editing induced by CRISPR-Cas9 in patient-derived fibroblasts. A plus strand single-guide RNA (sgRNA, bold underline) sequence targeting BMPR2 exon 8 in the region that corresponds to G to A missense mutation site was designed adjacent to a Protospacer Adjacent Motif (PAM, bold upper line). Precise cleavage of the third nucleotide (an arrowhead) from PAM by RNA-guided Cas9 induce double-strand breaks (DSB) in the BMPR2 mutant allele. The DSB is repaired by one of two general repair pathways: homology directed repair (HDR) and non-homologous end joining (NHEJ).

FIG. 4a shows that HDR generates a specific single nucleotide modification by using a knockin donor template (120 mer) with synonymous mutations (CTC, underline) and wildtype G nucleotide (upper line).

FIG. 4b shows that NHEJ-mediated DSB repair frequently induces nucleotide insertions or deletions at the DSB site of BMPR2 mutant allele that causes amino acid insertions, deletions, or frameshift mutations leading to premature stop codons within the open reading frame of BMPR2 gene.

FIG. 4c shows that patterns of deep sequencing reads induced by sgRNA of BMPR2 mutant allele. The efficiency of HDR from the pool of deep sequencing reads was about 1.63%. Indels of the total reads were about 53.6%.

FIGS. 5a and 5b are photos showing results of staining for detection of osteogenic differentiation. Duplicate samples (#1, 2) of normal, patient-derived cells, and CRISPR-modified cells (knock-out #3, #5, and knock-in #203) were incubated in 2% low serum medium at 37° C. with 5% CO₂ and 3% O₂ for 5 days and were performed for ALP staining to detect alkaline phosphatase, expressed in early osteogenic differentiation (FIG. 5a), and normal, patient-derived cells, and CRISPR-modified cells were maintained in 2% low serum medium without or with BMP2 or BMP4 (50 ng/ml) for 21 days and followed by Alizarin Red S staining to sense calcium deposits in late osteogenic differentiation (FIG. 5b).

FIGS. 6a and 6b are graphs showing mRNA level of TGF-β signaling target genes. To examine mRNA expression pattern of TGFβ signaling regulated by FOP-causative variants, empty vector (EV), BMPR2-wildtype, or BMPR2 E376K (FIG. 6a), EV, ACVR1-wildtype, or ACVR1 R206H (FIG. 6b) construct was transiently transfected into HEK293T cells. Expression level of TGFβ target genes including PAI-1, PDGFB, and THBS-1 was measured by RT-qPCR.

FIGS. 7a and 7b are photos showing that depletion of type I or type II TGF-β receptors by siRNA in BMPR2 E376K variant results in repression of SMAD activation.

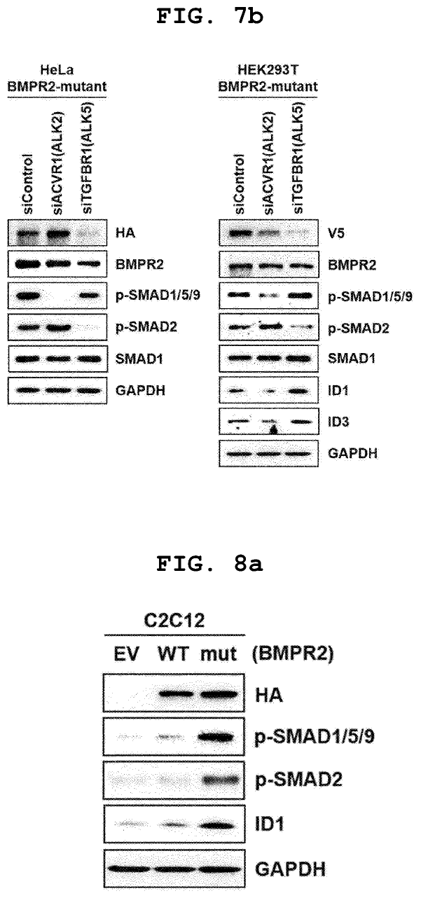

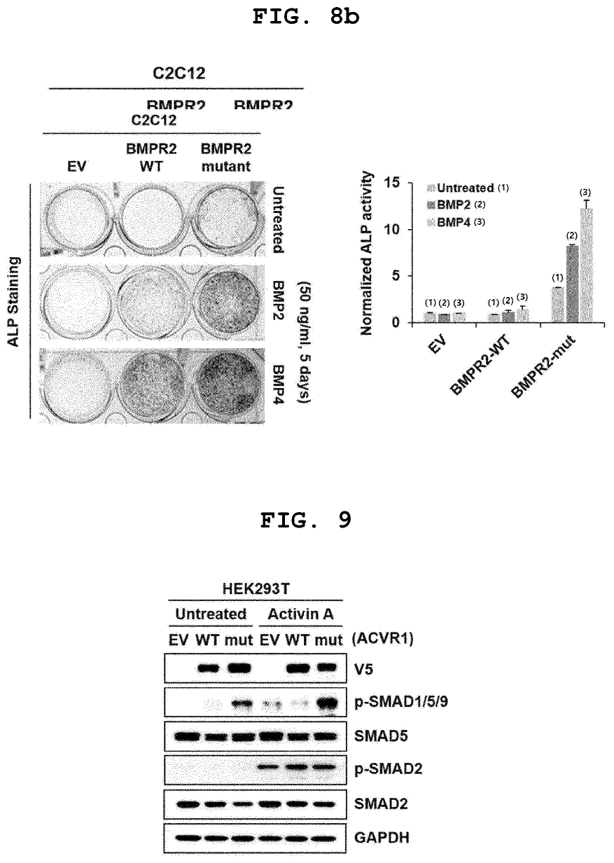

In FIG. 7a, HeLa and U2OS cells stably expressing empty vector, HA-BMPR2-wildtype, or HA-BMPR2-mutant established by lenti-viral transduction were treated with siRNA for BMPR2 using a manner of reverse and forward transfection. Protein expression of SMAD1/5/9 phosphorylation from cell lysates was analyzed by western blotting. The asterisk indicates the cross-reacting band.

In FIG. 7b, HeLa and HEK293T cells expressing BMPR2 E376K variant were transfected with siRNA for ACVR1 (ALK2) or TGFBR1 (ALK5), a type I receptor and then phosphorylation of SMAD1/5/9 and SMAD2 were detected by western blotting.

FIGS. 8a and 8b are photos and graphs showing osteogenic differentiation ability of BMP/SMAD signal hyper-activated by BMPR2 E376K variant in C2C12 cells.

In FIG. 8a, C2C12 cells stably expressing empty vector, HA-BMPR2-wildtype, or HA-BMPR2-mutant construct by lenti-viral transduction displayed protein expression for phosphorylation of SMAD1/5/9 and SMAD2 using western blotting, and in FIG. 8b, to differentiate from C2C12 cells to osteoblasts, cells were incubated in 2% low serum medium without or with BMP2 or BMP4 (50 ng/ml) at 37° C. with 5% CO₂. After 5 days, ALP staining was performed. For quantitation of ALP activity, lysates from osteogenic differentiated cells for 5 days were incubated 0.67 M pNPP solution for 30 minutes at 37° C. and then the reaction was immediately followed by analyzing in absorbance at 405 nm.

FIG. 9 is a photo showing protein level of SMAD phosphorylation by activin A treated in FOP causative mutant cells. HEK293T cells overexpressing empty vector, V5-ACVR1-wildtype, or V5-ACVR1 R206H were treated with activin A (50 ng/ml) for 5 minutes and then phosphorylation of SMAD1/5/9 and SMAD2 was confirmed through western blotting.

### DETAILED DESCRIPTION

Hereinafter, preferred embodiments of the present invention will be described in detail. Before describing the present invention, it should be understood that the terms and words used in the specification and the appended claims should not be construed as limited to general and dictionary meanings but interpreted based on the meanings and concepts corresponding to technical aspects of the present invention on the basis of the principle that the inventor is allowed to define terms appropriately for the best explanation for the invention.

Therefore, embodiments described in the specification and the example illustrated in the accompanying drawings herein is just a mere example for the purpose of illustrations only, not intended to represent all the technical aspects of the embodiment, the scope of the invention, so it should be understood that various equivalents and modifications thereof could be made at the time of filing.

Fibrodysplasia ossificans progressiva (FOP) is a rare autosomal dominant skeletal disorder characterized by progressive heterotopic ossification within soft connective tissues. All the patients presenting with clinical features of FOP so far have been identified to carry heterozygous gain-of-function mutations in the activin A type I receptor (ACVR1) gene.

With respect to this, the present invention presents a gain-of-function mutation in a bone morphogenetic protein type 2 receptor (BMPR2) gene encoding BMPR2 in a patient with a FOP-like phenotype.

A 16-year-old boy had subcutaneous migrating modules on the scalp at age 3 and developed a series of flare-ups and subsequent soft tissue ossification initiated from the neck and back to the extremities starting at 6 years of age. Whole exome sequencing revealed a heterozygous de novo mutation of BMPR2, c.1126G>A (p.E376K), which was located in the highly conserved kinase domain of BMPR2. Constitutive activation of BMP signaling was detected in the patient-derived dermal fibroblasts, which was abrogated upon CRISPR/Cas9-mediated BMPR2 silencing. Consistently, ectopic expression of the BMPR2-E376K mutant in other cell lines induces SMAD1/5/9 phosphorylation, even in the absence of BMP ligands. At the cytological level, the patient-derived cells were positive for alkaline phosphatase expression and calcium accumulation, both of which were abolished by treatment with dorsomorphin, a BMP signaling inhibitor. These findings indicate that the BMPR2-E376K mutation causes a phenotype of progressive heterotopic ossification, similar to that of constitutively active ACVR1 mutation.

Hereinafter, the present invention will be described in detail with reference to Examples.

A Case of FOP

A 16-year-old boy presented with flare-ups of the left pectoral region after treatment for dental caries. The patient was a product of a normal full-term pregnancy of a healthy Korean couple. Birth weight was 2.98 kg, and no perinatal problems were encountered. Motor and cognitive development was within the normal range. Subcutaneous migrating nodules were noted over the scalp at age 2 and over the posterior neck at age 4. Flare-ups and stiffness of the neck and back developed starting at age 6, and then progressed to the extremities. Physical examination revealed a completely stiff neck, back, and right shoulder. The upper left and lower right extremities maintained a functional range of joint motion. The feet and toes appeared normal.

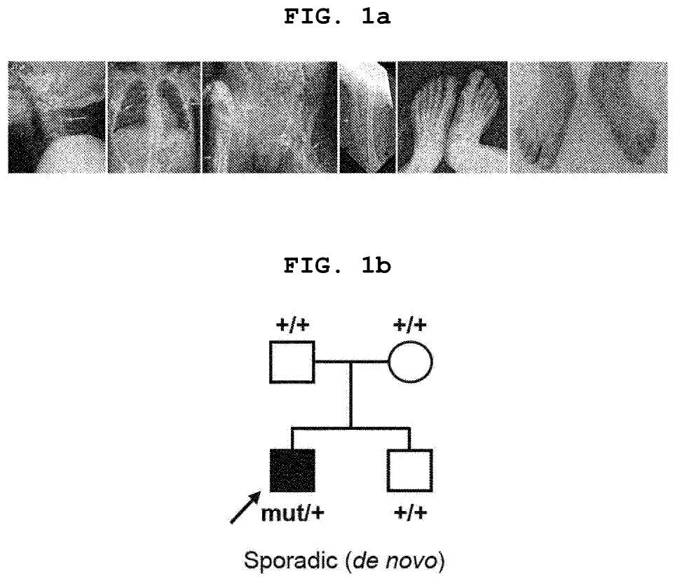

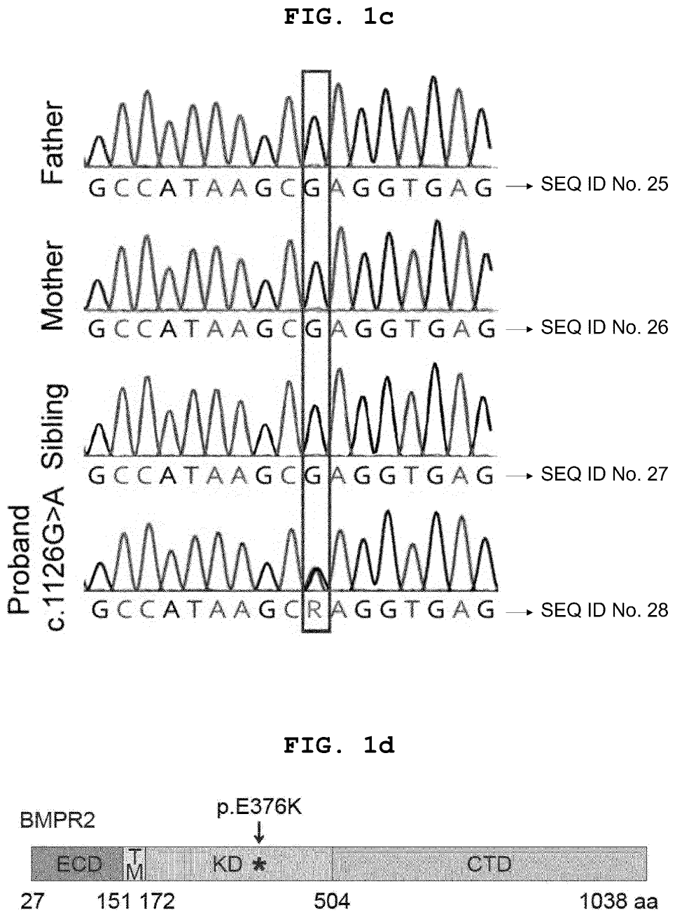

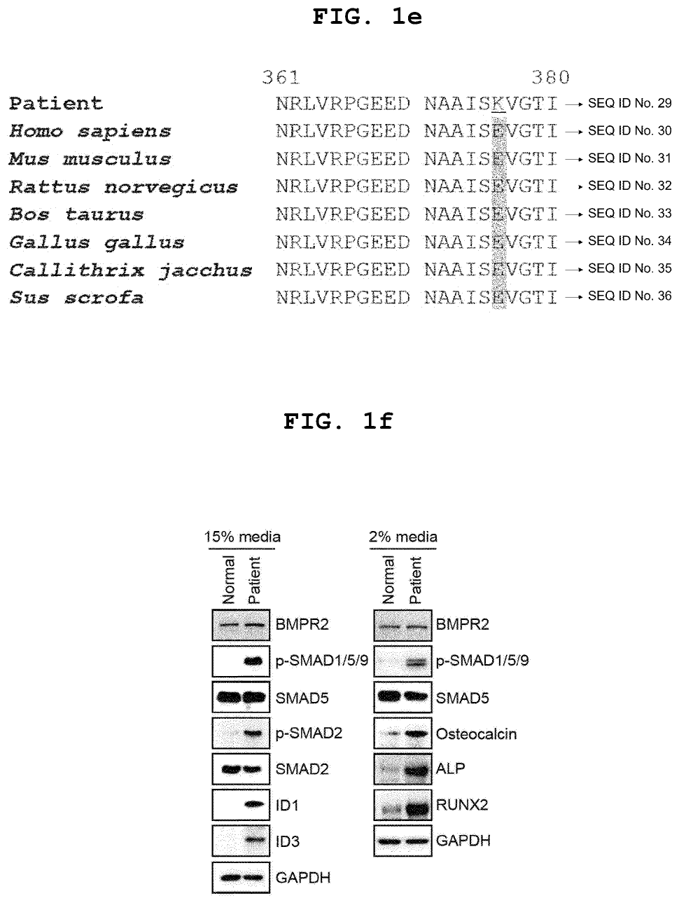

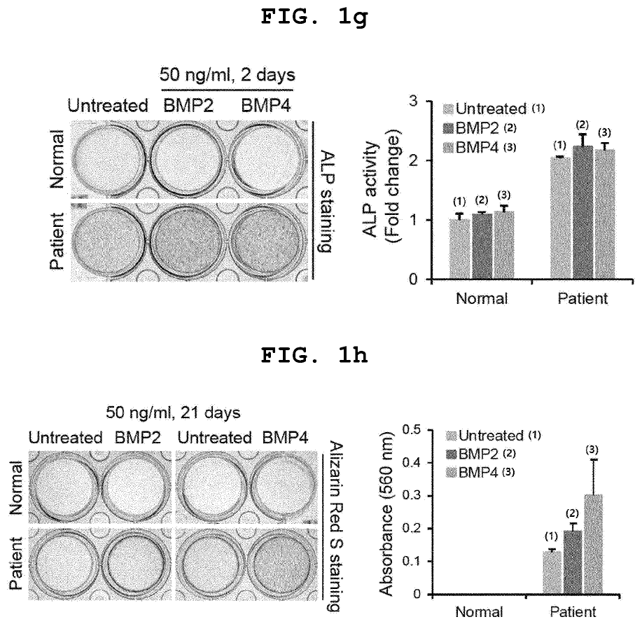

At age 22, the patient's height was 145 cm (z<−4) and weight was 57 kg. The whole neck and back, both shoulders and hips, and the left knee were completely fixed. Both elbows maintained only 10 to 30 degrees of flexion-extension motion, and the right knee maintained 80 degrees of motion. Radiographic examination showed heterotopic ossifications in the back muscles, periscapular muscles, peripelvic and thigh muscles (see FIG. 1a). Ankylosis of the posterior column of upper cervical vertebrae was also noted. It was noteworthy that there was no anomaly in the toes (see FIG. 1a). The patient recovered uneventfully from laparotomy for pyloric stenosis in childhood. At age 19, he developed severe dizziness. Brain MRI revealed multifocal gadolinium-enhanced tumors involving the suprasellar area, septum pellucidum, and medulla oblongata. They were clinico-radiologically diagnosed as mixed germ cell tumors because the serum beta-hCG level was moderately elevated. After radiation therapy, the tumors disappeared, and the patient has remained in complete remission for 3 years, with diabetes insipidus as its sequelae.

Methods

Genomic DNA was obtained from the proband, the sibling and his parents, and dermal fibroblast cells were derived from the proband, after obtaining written informed consent. The institutional Review Board of the Seoul National University Hospital, Seoul, South Korea, approved this study.

(1) Cell Culture, DNA Construction, Mutagenesis and FOP Cell Line Establishment

Patient-derived dermal fibroblasts and BJ (Normal) cells were grown in high-glucose and no-glutamine DMEM (GIBCO, Cat #10313) supplemented with 15% fetal bovine serum (FBS, GIBCO), Glutamax™ (GIBCO, Cat #35050-061) and non-essential amino acid (GIBCO, Cat #11140-050) and penicillin and streptomycin (GIBCO, 15140-122). Fibroblasts were incubated in 5% CO₂ and 3% O₂ at 37° C. BJ foreskin fibroblasts were obtained from ATCC. HEK293T, HeLa and U2OS cells were grown in high-glucose DMEM (GIBCO, Cat #11965) supplemented with 10% FBS (GIBCO) and 1× penicillin and streptomycin (GIBCO, Cat #15140-122) and they were incubated in 5% CO₂ at 37° C. C2C12 myoblasts were cultured in high-glucose, glutamine and sodium pyruvate DMEM (GIBCO, Cat #11995) supplemented with 15% FBS and 1× penicillin and streptomycin at 37° C. in 5% CO₂ humidified atmosphere. Undifferentiated C2C12 cells were sparsely maintained in a polystyrene cell culture dish to prevent myogenesis induced by cell contact. C3H10T1/2 fibroblasts were cultured in high-glucose, glutamine and sodium pyruvate DMEM (GIBCO, Cat #11995) supplemented with 10% FBS and 1× penicillin and streptomycin and they were incubated in 5% CO₂ at 37° C. Primary patient-derived fibroblasts and BJ cells were immortalized by expressing the catalytic subunit of human telomerase (hTERT) through lentiviral transduction and transformed by the human papilloma virus E6 and E7 protein through retroviral transduction. BMPR2 cDNA was obtained from addgene. BMPR2 cDNA was cloned to EcoRI restriction sites in pcDNA6/V5-HisABC vector using In-Fusion HD Cloning kits (Takara, Cat #638920) and pDONR223 BP vector and later pHAGE-HA-FLAG LR vector using Gateway cloning system (Thermo Fisher Scientific). C.1126G>A BMPR2 mutation was generated by QuikChange II XL Site-Directed Mutagenesis Kit (Agilent Genomics) with the following primer; BMPR2-F (SEQ ID No. 1) 5′-GGGGACAAGTTTGTACAAAAAAGCAGGCTTAATGACTTCCTCGCTGCAGCGGC-3′, BMPR2-R (SEQ ID No. 2) 5′-GGGGACCACTTTGTACAAGAAAGCTGGGTCTCACAGACAGTTCATTCC-3′. Cell lines stably expressing BMPR2 or BMPR2 mutants were generated by lentiviral transduction as previously described (see non-patent document 26).

(2) Osteogenic Differentiation

BJ and patient-derived dermal fibroblasts (8×10⁴ cells/well) were seeded into 24-well cell culture plates and then cultured in DMEM (GIBCO, Cat #10313) supplemented with 15% FBS, 1% Glutamax, 1% non-essential amino acid and 1% penicillin-streptomycin at 37° C. with 5% CO₂ and 3% O₂. To induce differentiation, growth medium was replaced into DMEM supplemented with 2% horse serum (GIBCO, Cat #16050) after cells reached 80-90% confluence. Cells were maintained without or with recombinant human BMP2 or BMP4 (R&D SYSTEMS) and replaced with fresh medium every 2-3 days for 2-21 days. C2C12 cells were seeded into 24-well cell culture plates at a density of 4×10⁴ cells/well. Cells were grown in DMEM (GIBCO, Cat #11995) supplemented with 15% FBS at 37° C. with 5% CO₂. Cells with 80-90% confluence were replaced by osteogenic differentiation DMEM (GIBCO, Cat #11995) containing 100 nM dexamethasone, 10 mM β-glycerophosphate, and 50 μM ascorbic acid-2-phosphate (all from Sigma) supplemented with 2% horse serum. C2C12 were treated with BMP2, BMP4, dorsomorphin (Sigma), or 5B431542 (Sigma) and maintained with replacement of fresh medium every 2-3 days for 3-21 days.

(3) Chondrogenic Differentiation

For chondrogenesis, C3H10T1/2 cells were cultured by micromass technique, high density dot culture. First, cells were resuspended in DMEM supplemented with 10% FBS and 1× penicillin-streptomycin at a concentration of 10⁷ cells/ml and a 10 μl droplet of the cell suspension was placed in the center of a well of 12-well cell culture plates followed by incubation at 37° C. and 5% CO₂. After 2 hours, 1 ml chondrogenic differentiation medium consisting of 1% FBS, 1% Insulin-Transferrin-Selenium (GIBCO), 0.1 μM dexamethasone, 0.17 mM ascorbic acid-2-phosphate, 0.35 mM proline (Sigma), and 0.15% glucose (Sigma) was added in each well and cells were maintained without or with human recombinant BMP2.

(4) Whole Exome Sequencing and DNA Analysis

Written informed consent was obtained from the affected individual. The Institutional Review Board of the Seoul National University Hospital, Seoul, South Korea approved the studies. Genomic DNAs were extracted from whole blood and sequencing libraries were prepared using Twist modular library preparation kits. SureSelect Human All Exon V5 baits (Agilent, Santa Clara, Calif.) covering all exon regions were used. Targeted sequencing was performed with 101 base pair (bp) paired-end reads on an Illumina HiSeq2500 platform (Illumina, San Diego, Calif.). Sequenced reads were aligned to human genome reference sequence (hg19) using Burrows-Wheeler Aligner (BWA) version 0.7.5a with the Maximum Entropy Method (MEM) algorithm. At the same time, the aligned reads were selected mapping phred quality score above 30, converted to binary alignment map (BAM) format and sorted ordering by genomic position using SAMTOOLS version 1.2. For high performance accurate variant calling, i) PCR duplicates reads were marked using MarkDuplicates of Picard tools version 1.127 (http://broadinstitute.github.io/picard/). ii) Insertion and deletion (Indel) realignment were performed with known Indels from Mills and 100G gold standard using RealignerTargetCreator and IndelRealigner of Genome Analysis Tool Kit (GATK) version 3.1-1. iii) Base quality score was recalibrated using machine learning model with known single nucleotide polymorphisms (SNPs) and Indels from dbSNP138, Mills and 1000 Genome Project phase I by BaseRecalibrator and PrinReads of GATK. Manipulated BAMs were simultaneously called and genotyped of single nucleotide variants (SNVs) and Indels by GATK UnifiedGenotyper uses a Bayesian genotype likelihood model. Variants were recalibrated with reference variants such as dbSNP138, Mills Indels, HapMap and Omni using GATK VariantRecalibrator and ApplyRecalibration. Variants were annotated various information using ANNOVAR described below: i) population database such as 1000 genome phase III, ExAC and KRGDB (http://coda.nih.go.kr/coda/KRGDB/), ii) disease database such as OMIM, sequencing database such as RefSegGene, iii) in silico predictive algorithms such as FATHMM, MutationAssessor, MutationTaster, SIFT, Polyphen, GERP and Phylop for interpretation and classification of variants following ACMG guideline. Classified pathogenic or likely pathogenic variants were confirmed by Sanger sequencing. Copy number variants (CNVs) were calculated using aligned read counts in target region by in-house relative comparison method. Detected and classified pathogenic CNVs were re-confirmed by array comparative genomic hybridization (array CGH) (see non-patent documents 27 to 31).

(5) CRISPR-Cas9 Mediated Gene Correction

Along with 10 μM single-stranded oligodeoxynucleotides (ssODN) donor template, 4 μg of S. pyogenes Cas9 (SpCas9) protein and 1 μg of guide RNA selectively targeting mutated allele of FOP patients were prepared as RNP complex and delivered into fibroblasts obtained from patients with Neon electroporator (Invitrogen). Target sequence (SEQ ID No. 3) for CRISPR-Cas9 is as follows; 5′-agataatgcagccataagcaagg-3′ (PAM sequence:underlined). To distinguish the corrected allele from wild type and to prevent the recurrent cleavage, donor template (SEQ ID No. 4) was designed as follows; 5′-ccatgaggctgactggaaatagactggtgcgcccaggggaggaagataatgcagccatCTCcga ggtgagtgtatacaaaaggtatcacactgatgtactttgaaatgataatttaatta (upper underlined: codon matched sense mutation, italic underlined: corrected base). 1 week after electroporation, frequency of properly corrected allele from pooled fibroblasts were analyzed by targeted deep sequencing. Based on that frequency, cells were dissociated and re-plated in 96-well plates at the density of ⅓ cell per well to obtain single cell colony. After single cell colony isolation, gDNAs of each clone were harvested and analyzed by targeted deep sequencing, and proper colonies were selected for further experiments.

(6) Targeted Deep Sequencing

To quantify Indel ratio and analyze the sequence after CRISPR-Cas9 treatments, target region was amplified with PCR primers hybridizing the target amplicon sequences with illumina barcode sequences by nested PCR. PCR products were purified, denatured by NaOH, and subjected to 2×250 paired-end sequencing with an Illumina MiSeq. Paired-end reads from MiSeq were analyzed by Cas-Analyzer (http://www.rgenome.com). PCR primers used in this experiment were as follows; hBMPR2 E7 F1 (SEQ ID No. 5): 5′-gcctccttttacagccctat-3′, hBMPR2 E7 R1 (SEQ ID No. 6): 5′-aactttacccttgcctcaaa-3′, hBMPR2 E7 dF1 (SEQ ID No. 7): 5′-ACACTCTTTCCCTACACGACGCTCTTCCGATCTacagcagaaatgtcctag, hBMPR2 E7 dR1 (SEQ ID No. 8) 5′-GTGACTGGAGTTCAGACGTGTGCTCTTCCGATCTctctttaccttaggtgat.

(7) Small Interfering RNA, siRNA

siRNAs were transfected twice into cells, first by reverse transfection and 24 hours later by forward transfection using Lipofectamine RNAiMAX reagent (Invitrogen) as suggested by the manufacturer's instructions. ACVR1 (ID #s974, s976), TGFBR1 (ID #s14071, s14073), and BMPR2 (ID #s2044, s2045, s2046) siRNAs were purchased from Thermo Fisher Scientific. Pools of two or three siRNAs were used with a final siRNA concentration of 25 nM.

(8) Luciferase Reporter Assay

293T cells were plated in the Falcon® 96-well white flat bottom tissue culture-treated microtest assay microplate (CORNING). In each well, 5,000 cells were plated in 100 μl 10% DMEM media. 24 hours after plating, cells were transfected with pcDNA-empty vector, pcDNA6/V5-HisA-wildtype BMPR2 or -mutant BMPR2, pGL3-BMP responsive elements-luciferase (hereafter pGL3-BRE-luc, offered from addgene plasmid #45126), and pNL1.1.TK internal control vector for the assay, using calcium phosphate transfection Kit (Invitrogen). The amounts of WT or mutant BMPR2, and pGL3-BRE-luc, and pNL1.1 from Nano-Glo® Dual-Luciferase® Reporter Assay Kit (Promega) were determined according to a protocol of calcium phosphate transfection from Clontech Laboratories; 50 ng of WT BMPR2 or mutant BMPR2 and pGL3-BRE-luc and 5 ng of pNL1.1.TK were used and then 2M Calcium Solution and sterile water were added in each DNA tube. The same volume of 2×HEPES-Buffered Saline (HBS) was added to Calcium-DNA mixture dropwise and incubated at room temperature. After 15 minutes, the transfection solution was carefully added to culture plate medium and maintained at 37° C. in a CO₂ incubator. The next day, the calcium phosphate-containing medium was removed from cells and replaced with fresh complete growth medium. A volume of One-Glo™ EX Luciferase assay Reagent was equally added to the culture medium volume to each well and placed on an orbital shaker at 300 rpm for 3 minutes. Luminescence was measured as integration times of 1 second by GloMax® Discover System (Promega). For measurement of NanoLuc® luciferase activity, a volume of NanoDLR™ Stop & Glo® Reagent was equally added to the original culture medium volume to each well and then luminescence was analyzed. The BRE reporter luminescence was normalized to NanoLuc® luciferase activity.

(9) Western Blotting and Immunoprecipitation

Cells were plated either in 60 mm or 100 mm plate with 70% confluency. The next day, plasmid DNA was transfected into HEK293T cells by Lipofectamine 2000. After 4 hours, cells were changed into serum free medium and treated with human recombinant activin A (R&D SYSTEMS) the next day. Cells were harvested and lysed by lysis buffer (50 mM Tris-HCl pH7.5, 150 mM NaCl, and 0.5% Nonidet P-40) containing a protease inhibitor cocktail (Roche) and quantified by Protein Assay Dye Reagent Concentrate (Bio-Rad) and NanoDrop (Thermo Fisher Scientific). Proteins were separated by 8-15% sodium dodecyl sulfate-polyacrylamide gel electrophoresis (SDS-PAGE) and gels were blotted onto polyvinylidene difluoride (PVDF) transfer membrane with 0.45 μm pore size (Merck Millipore). Blots were blocked in 1×PBS with 0.1% Tween-20 (Sigma) containing 5% Difco™ Skim Milk (BD) for 1 hour at room temperature and incubated with anti-V5-Tag (Invitrogen, #R960-25), anti-phospho-SMAD1/5/9 (Cell Signaling Technology, #13820), anti-SMAD1 (CST, #6944), anti-SMAD5 (CST, #12534), anti-phospho-SMAD2 (CST, #3108), anti-SMAD2 (CST, #5339), anti-SMAD4 (CST, #9515), anti-ID1 (SANTA CRUZ BIOTECHNOLOGY, sc-488), anti-ID3 (SCBT, sc-490), anti-HA (Covance, MMS-101R), anti-50X9 (CST, #82630), anti-BMPR2 (CST, #6979), anti-Osteocalcin (Merck Millipore, AB10911), anti-Alkaline Phosphatase (abcam, ab108337), anti-RUNX2 (SCBT, sc-10758) and anti-GAPDH (SCBT, sc-25778) as a loading control at 4° C. for overnight. After blots were washed four times in 1×PBST for 1 hour at room temperature, anti-mouse secondary (Jackson ImmunoResearch, 115-035-003) or anti-rabbit secondary (Jackson ImmunoResearch, 111-035-003) was used at 1:2500 for 2 hours at room temperature and then bands were detected by enhanced chemiluminescence solution (Bio-Rad) using ChemiDoc System (Bio-Rad). The band image was analyzed with Image Lab™ Software (Version 5.2.1, Bio-Rad).

For immunoprecipitation, transiently transfected HEK293T cells were lysed and sonicated in lysis buffer at 4° C. Crude lysates cleared by centrifugation at 15,000 rpm at 4° C. for 20 minutes. Supernatants were incubated with Monoclonal Anti-HA-Agarose antibody (Sigma) for 2 hours at 4° C. Immunocomplex was washed five times with lysis buffer and then SDS-PAGE and western blotting were performed.

(10) Real-Time Quantitative Reverse Transcription PCR

Total RNA of the cells was extracted using RNeasy Mini Kit and QIAshredder (QIAGEN) and quantified using NanoDrop instrument. 1 μg of total RNA was used to cDNA synthesis using a SuperScript III First-Strand Synthesis System (Invitrogen). Gene expression was quantified by 2× qPCRBIO SyGreen Blue Mix Lo-ROX (PCRBIOSYSTEMS) performed on LightCycler® 96 (Roche). Quantification cycle (Cq) values of samples were analyzed by LightCycler® 96 Application Software (Version 1.1). Gene-specific primers are shown in Table 1 below.

TABLE 1

| sample | | Sequence 5′ → 3′ | Notes |
|---|---|---|---|
| mCol2a1 | sense | CCTCCGTCTACTGTCCACTGA | SEQ ID No. 9 |
| | antisense | ATTGGAGCCCTGGATGAGCA | SEQ ID No. 10 |
| mCol10a1 | sense | AACAGGTATGCCCGTGTCTG | SEQ ID No. 11 |
| | antisense | TCATCAAATGGGATGGGGGC | SEQ ID No. 12 |
| mAggrecan | sense | TGGCTTCTGGAGACAGGACT | SEQ ID No. 13 |
| | antisense | TTCTGCTGTCTGGGTCTCCT | SEQ ID No. 14 |
| mGapdh | sense | CATGTTCCAGTATGACTCCACTC | SEQ ID No. 15 |
| | antisense | GGCCTCACCCCATTTGATGT | SEQ ID No. 16 |
| hPAI-1 | sense | TCCTGGTTCTGCCCAAGTT | SEQ ID No. 17 |
| | antisense | CCAGGTTCTCTAGGGGCTTC | SEQ ID No. 18 |
| hPDGFB | sense | CTGGCATGCAAGTGTGAGAC | SEQ ID No. 19 |
| | antisense | CGAATGGTCACCCGAGTTT | SEQ ID No. 20 |
| hTHBS-1 | sense | CAATGCCACAGTTCCTGATG | SEQ ID No. 21 |
| | antisense | TGGAGACCAGCCATCGTC | SEQ ID No. 22 |
| hGAPDH | sense | AGCCACATCGCTCAGACAC | SEQ ID No. 23 |
| | antisense | GCCCAATACGACCAAATCC | SEQ ID No. 24 |

(11) Alizarin S Staining (Mineralization Assay)

The mineralization was determined by staining with Alizarin Red S at 21 days after osteogenic differentiation. For preparation of solution, 2 g Alizarin Red S (Sigma) was dissolved in 100 ml distilled water and then adjusted to pH4.3 with HCl or NH₄OH. Differentiated cells were carefully washed with PBS and fixed with 4% paraformaldehyde (Sigma). After 30 minutes carefully washed the cells with distilled water followed by prepared stain solution was enough added to the cells for 45 minutes at room temperature in the dark. The cells were washed four times with distilled water and carefully aspirated. The differentiated cells are stained darker red with calcium deposits. After photography using digital camera (Nikon), the stained cells were lysed with 10% cetylpyridinium chloride (sigma) dissolved in 10 mM sodium phosphate buffer (1 M NaH₂PO₄ monobasic and 1 M Na₂HPO₄ dibasic, pH7.0) and then quantified at 560 nm using a GloMax® Discover System.

(12) Alkaline Phosphatase (ALP) Staining and Activity

For detection of alkaline phosphatase, cells were firstly cultured with osteogenic differentiation media for 2 or 3 days. Cells were cautiously washed with PBS and then fixed with 4% paraformaldehyde. After 1 minute, cells were rinsed with Washing Buffer (0.05% Tween 20 in PBS), subsequently treated with substrate solution which was dissolved one BCIP/NBT tablet (Sigma) in 10 ml distilled water. For staining, the cells were incubated at room temperature in the dark for 10 minutes monitoring staining progress every 2-3 minutes. Carefully aspirated the substrate solution and rinsed the cell with Washing Buffer. The higher alkaline phosphatase, the more intense the dark blue-violet. For ALP activity, cultured cells were washed with PBS and lysed with cold alkaline phosphatase reaction buffer (1 M Diethanolamine and 0.5 mM Magnesium Chloride, pH9.8, Sigma). Lysates were incubated in 0.67 M p-Nitrophenyl Phosphate (pNPP) solution (Sigma) for 30 minutes at 37° C. continuing the reaction was immediately followed by monitoring in absorbance at 405 nm. Total protein was measured by using a Micro-BCA protein assay kit (Thermo Fisher Scientific) and read at 560 nm using a GloMax® instrument. The enzymatic ALP activity was normalized to the protein content of the samples.

(13) Alcian Blue Staining

To visualize ability of chondrogenesis, stain solution (pH1.0) was prepared with 1 g Alcian blue 8GX (Sigma) in 100 ml 0.1 M HCl. Cells were fixed with 4% paraformaldehyde in PBS for 20 minutes at room temperature and then rinsed 3 times with PBS. Alcian blue solution was used to stain the cells at room temperature in the dark. Next day, cells were washed once with 0.1 M HCl and twice with PBS. After taking a picture, the dye was extracted by Guanidine-HCl (Sigma) for 2 hours at room temperature and then read in absorbance at 600 nm using a GloMax® instrument.

Results

The clinical manifestations were consistent with FOP, except for the absence of a big toe anomaly (see FIG. 1a). Full sequencing of the coding region of ACVR1 (MIM #102576) of the proband showed only two silent sequence variations, both of which were previously reported in normal population: c.270C>T (NCBI dbSNP rs2227861) and c.690G>A (rs1146031). Then, whole exome sequencing was performed on the genomic DNA from the proband and a heterozygous variation in BMPR2 (MIM #600799), c.1126G>A (p.E376K) was identified. Sanger sequencing and restriction enzyme digestion confirmed the sequence variation in the proband and absence in the genome of the parents and the sibling (see FIGS. 1B to 1c). The BMPR2-E376K mutation is located in the kinase domain (see FIG. 1d) (see non-patent document 13) where the sequence is highly conserved among species (see FIG. 1e). The BMPR2 sequence variation was not found in the general populations of the 100 genome, ExAC, dbSNP, or Exome Sequencing Project databases.

To gain insight into the molecular basis of the disease phenotype, BMPR2 protein changes were determined by immunoblotting the lysates prepared from dermal fibroblasts obtained from the patient's skin and a normal control. As shown in FIG. 1f, lysates from the patient-derived cells showed constitutive phosphorylation of SMAD1/5/9, and the resultant increased expression of ID1 and ID3, which was not the case in the lysates prepared from the normal BJ control cells (see FIG. 1f). Interestingly, it was noticed that SMAD2 was also phosphorylated in these cells. In addition, patient-derived cells cultured in the differentiation media expressed a group of proteins (see non-patent document 14) implicated in bone development including osteocalcin (OCN), alkaline phosphatase (ALP), and RUNX2, even in the absence of BMP treatment (see FIG. 1f, right panel). These molecular changes were reflected at the cellular level. ALP expression is known as a molecular marker for bone development (see non-patent document 15). As expected, the patient-derived cells were positive for ALP staining and ALP activity while normal control cells barely express the ALP (see FIG. 1g). Furthermore, it was found that calcium was accumulated in the culture of the patient-derived cells, which was determined by alizarin red S staining after 21 days in culture (see FIG. 1h) (see non-patent document 16). Taken together, these findings strongly suggest that the BMPR2-E376K variant constitutively activates BMP signaling, even in the absence of BMP treatment.

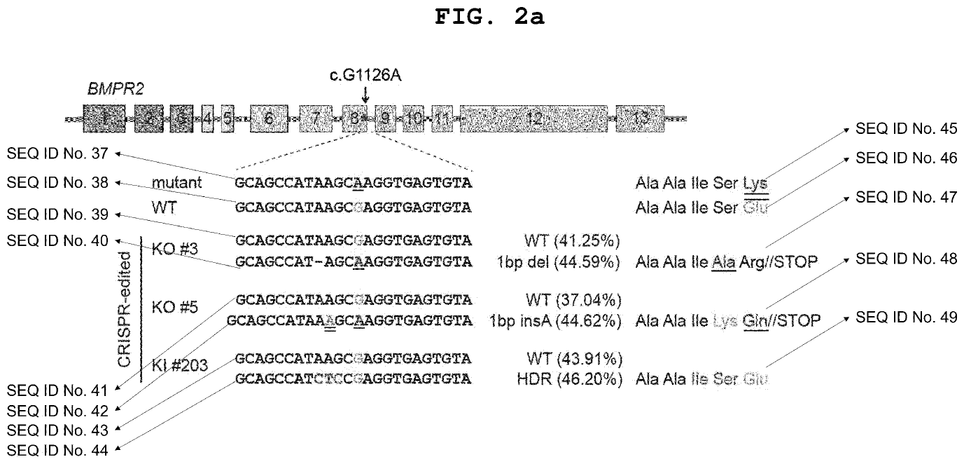

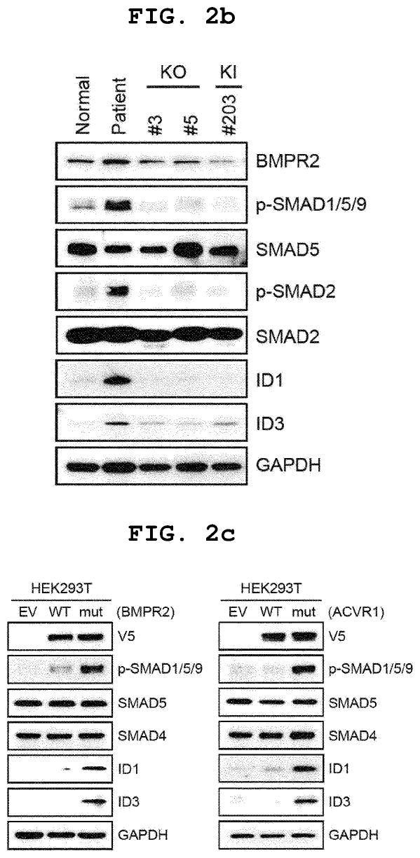

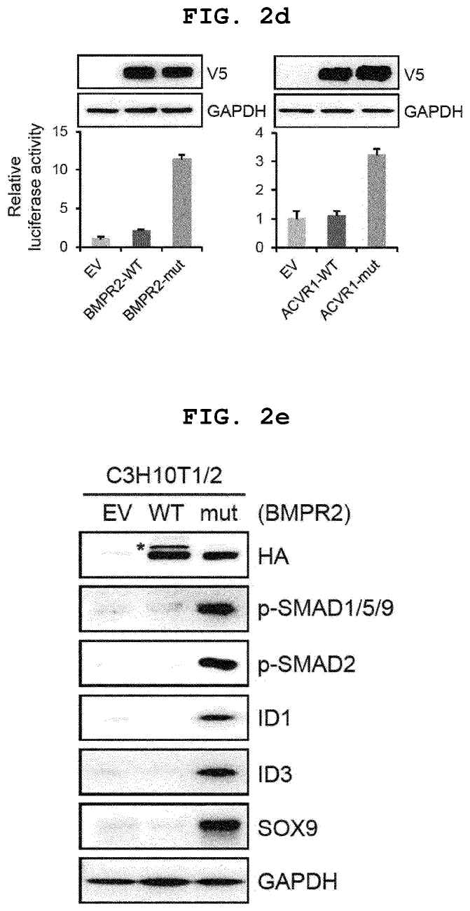

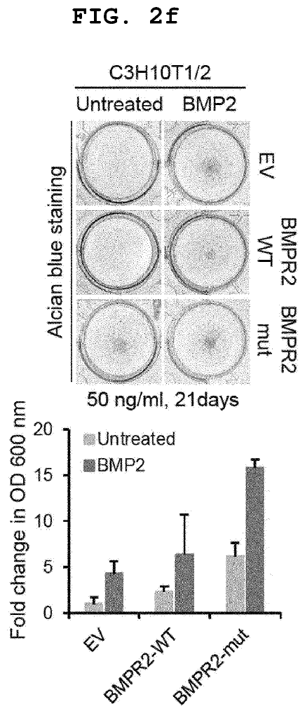

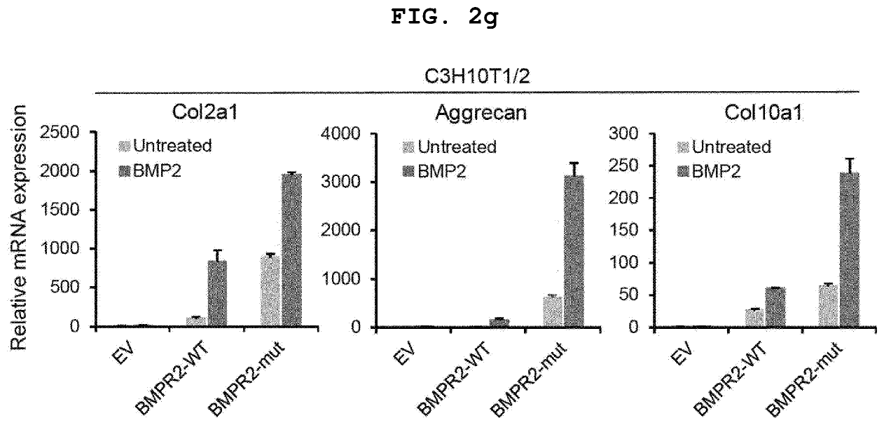

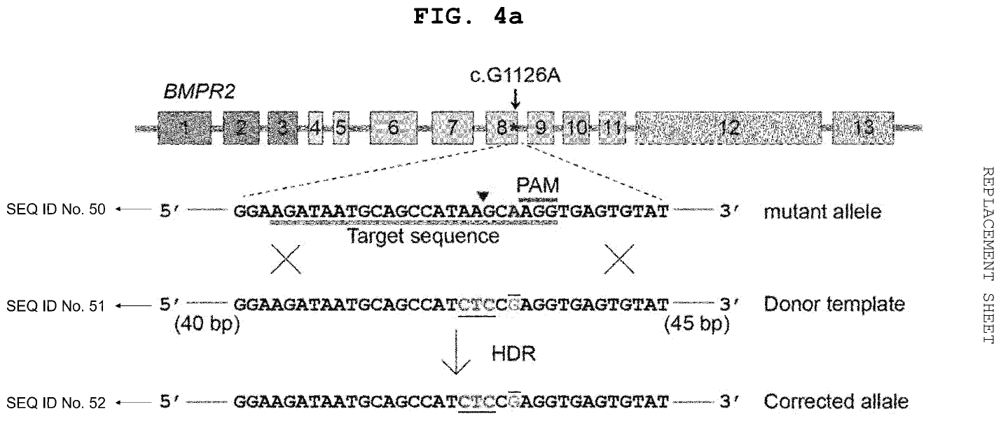

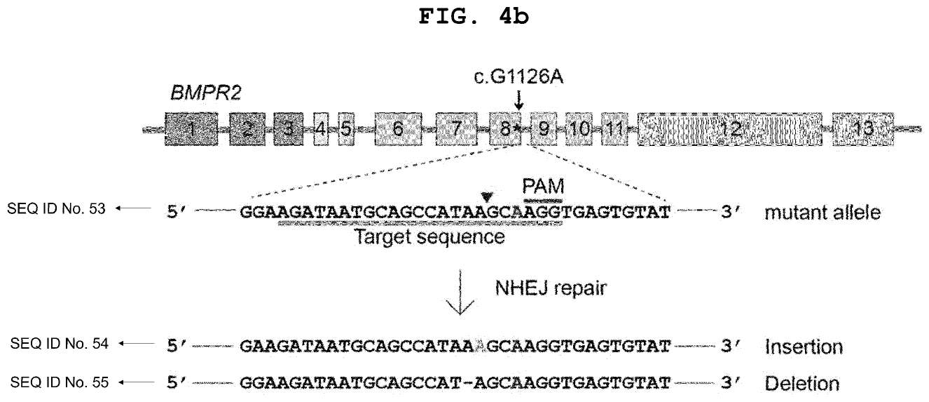

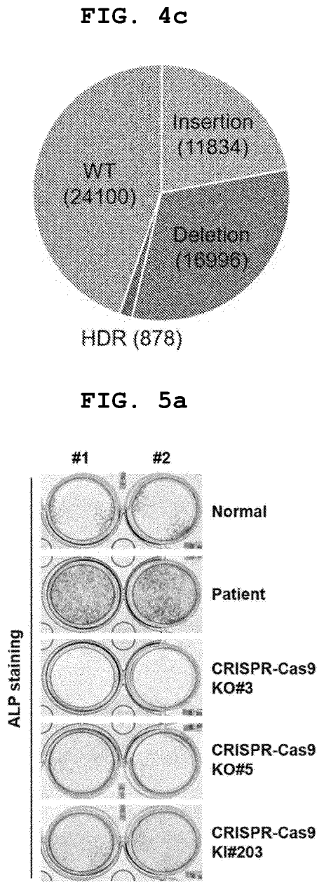

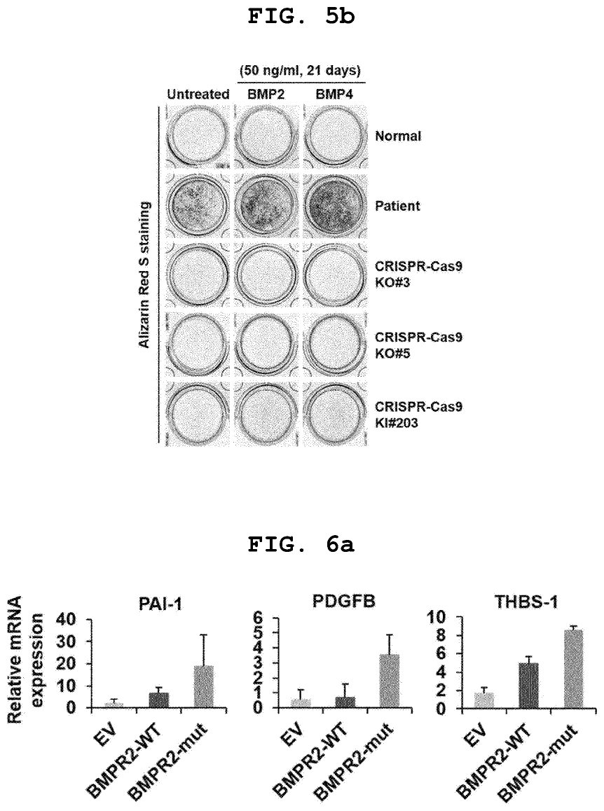

Functional validation of pathogenicity of the potential causative mutation is critical. DNA sequence analysis suggested the BMPR2-E376K variant was functionally dominant, and therefore it was hypothesized that if the mutated BMPR2 allele was deleted, the hyperactivated BMP signaling would return to normal. To this end, the mutated BMPR2 allele was first deleted using CRISPR-Cas9 in the patient-derived cells. At the same time, the c.1126G>A variant was reverted back to the WT sequence using CRISPR-Cas9 knock-in methods. A large number of single clones were carefully isolated and sequences of the BMPR2 gene in individual clones were determined using a MiSeq system (see FIG. 2a and FIG. 4). It was found that both knock-out and knock-in clones lost the SMAD1/5/9 phosphorylation (see FIG. 2b), ALP staining, and calcium deposition (see FIG. 5), demonstrating that the BMPR2-E376K variant is causative for the constitutive BMP activation and resultant outcomes.

Next, the inventors reasoned that ectopic expression of the BMPR2-E376K variant in different cell types would recapitulate the molecular and cellular changes caused by expression of the functionally dominant genomic variant. To test this, an empty vector, WT BMPR2, or BMPR2-E376K were expressed in HEK293T cells, respectively (see FIG. 2c) and it was found that only the expression of BMPR2-E376K led to hyperphosphorylation of SMAD1/5/9 and expression of ID1 and ID3. In addition, to test if enhanced BMP signaling due to the expression of BMPR2-E376K results in BMP response gene expression, a transcriptional reporter assay system in which luciferase expression is controlled by BMP-responsive elements was established (see non-patent document 7). As expected, it was noticed that expression of BMPR2-E376K in HEK293T cells led to significant enhancement of luciferase activity (see FIG. 2d). In the same experimental setting, similar results were obtained when the known causative ACVR1-R206H mutant was expressed in HEK293T cells (see FIGS. 2c to 2d, right panels). Taken together, these findings demonstrate the dominant gain-of-function features of the BMPR2-E376K mutation.

Endochondral heterotopic ossification in FOP lesions involves chondrogenic differentiation from mesenchymal stem cells (MSCs) due to the enhanced BMP signaling, which later turns into mature bone tissue (see non-patent document 14). As the BMPR2-E376K mutant stimulates BMP signals in the absence of BMP ligands, the inventors hypothesized that the BMPR2-E376K variant might force the MSCs to become chondrocytes. To test this idea, an empty vector, WT BMPR2, or BMPR2-E376K were individually expressed in mouse MSC cell line C3H10T1/2. As shown in FIG. 2e, it was found that temporal expression of BMPR2-E376K led to phosphorylation of SMAD1/5/9 and expression of its downstream effectors including ID1, ID3, and SOX9. In addition, it was found that C3H10T1/2 cells expressing BMPR2-E376K were forced to become chondrocytes, based on the Alcian blue staining (see FIG. 2f). Indeed, later in culture, expression of the BMPR2-E376K mutant showed enhanced expression of COL2A1, COL10A1, and Aggrecan, all of which are highly expressed in osteoblasts (see FIG. 2g) (see non-patent document 17). Altogether, these findings demonstrate that BMPR2-E376K is causative for the FOP phenotype and is a typical gain-of-function mutation.

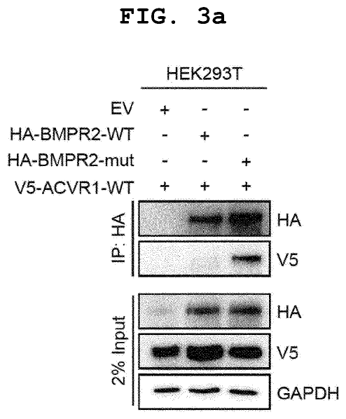

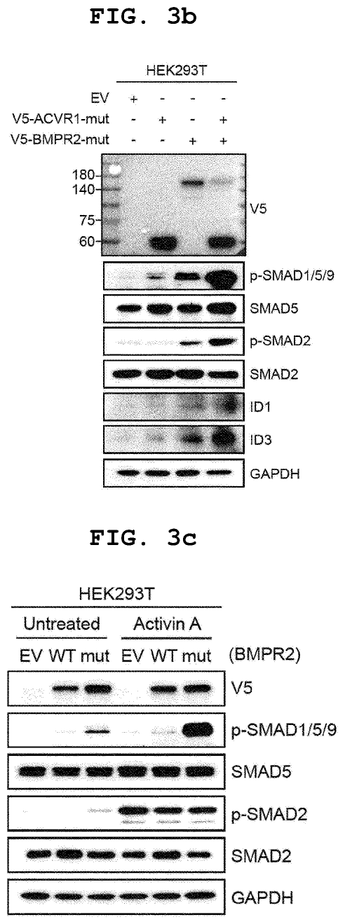

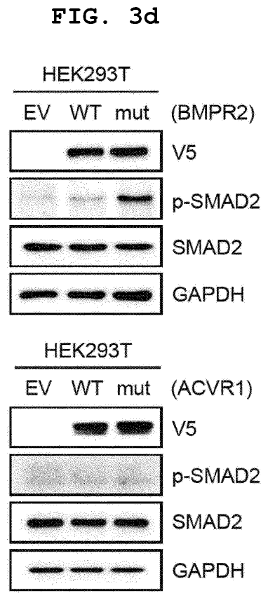

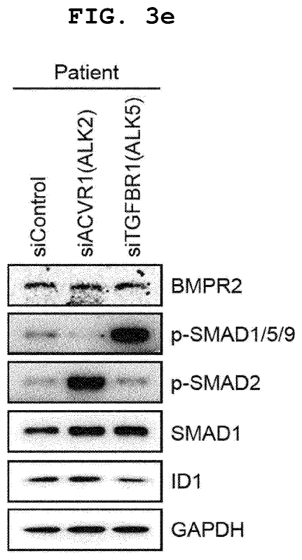

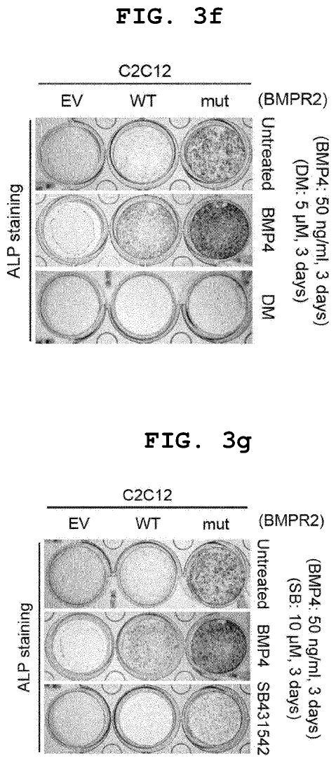

BMP signaling is activated in the presence of BMP ligands, which leads to engagement of type I and type II receptors. The same is true in other cellular receptor systems, and it was reported that gain-of-function mutations in other receptors cause forced association of receptor partners, resulting in constitutive signal transduction. In an attempt to understand the molecular consequences of the gain-of-function BMPR2-E376K mutation, it was tested if the BMPR2-E376K mutant can associate with ACVR1 in the absence of BMP ligands. To this end, V5-tagged ACVR1 was transiently expressed together with either WT or mutant HA-tagged BMPR2. Surprisingly, it was found that ACVR1 co-immunoprecipitated with BMPR2-E376K, whereas WT BMPR2 did not associate with ACVR1 in the absence of BMP ligands (see FIG. 3a), suggestive of a molecular basis for constitutive activation of BMP signaling by the BMPR2-E376K mutant. In addition, combined expression of the ACVR1-R206H and BMPR2-E376K variants showed additive effects of SMAD1/5/9 phosphorylation (see FIG. 3b), indicating that BMPR2-E376K variant has a different molecular mechanism of BMP signal activation than ACVR1-R206H. However, similar to the case of ACVR1-R206H mutation (see FIG. 9) (see non-patent documents 11 and 12), it was noticed that treatment with activin A, a non-osteogenic member of the TGF-beta superfamily stimulating SMAD2/3 phosphorylation (see non-patent documents 11, 18 and 19), enhances SMAD1/5/9 phosphorylation in cells expressing BMPR2-E376K, but not empty vector or WT BMPR2 (see FIG. 3c). Considering the fact that activin A is an obligatory factor for the FOP phenotype, this finding further supports idea that the BMPR2-E376K mutation is causative for FOP, although the molecular mechanism remains elusive.

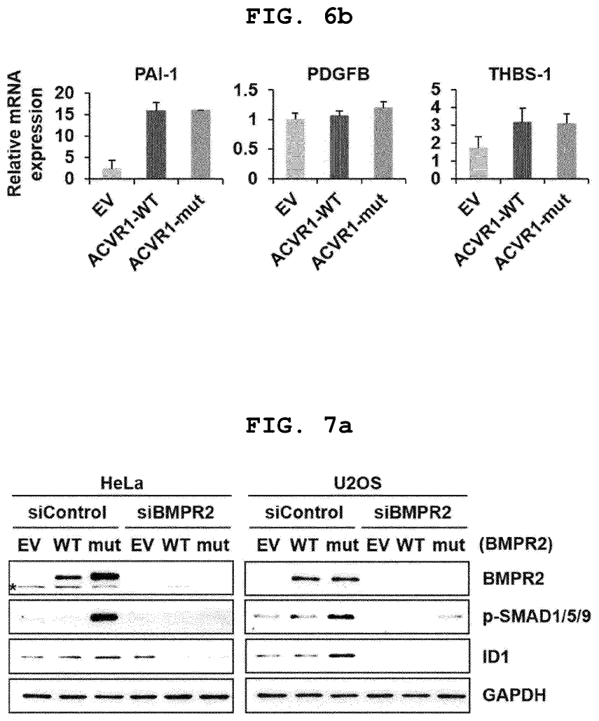

Unlike ACVR1-R206H, it was observed SMAD2 phosphorylation was enhanced in the patient-derived cells (see FIG. 1f), and also in HEK293T cells expressing BMPR2-E376K, which is not the case in the cells expressing the ACVR1-R206H variant (see FIG. 3d). Quantitative mRNA detection confirmed that the expression of the genes downstream is increased (see FIG. 6), implying that SMAD2 phosphorylation due to the BMPR2-E376K variant is indeed functional. In order to specify the type I TGF-beta receptor responsible for SMAD2 activation in the BMPR2-E376K background, individual type I receptors in the patient-derived cells were depleted, and it was found that depletion of TGFBR1 led to abrogation of the SMAD2 phosphorylation in patient-derived cells (see FIG. 3e) and also other cells stably expressing the BMPR2-E376K mutant (see FIG. 7). To understand the functional roles of SMAD2 activation in FOP lesions in the cells expressing BMPR2-E376K, mouse myogenic C2C12 cells were used, which are derived from the target tissues of FOP pathogenesis. Through lentiviral transduction, the inventors established C2C12 cell lines which stably express empty vector, WT human BMPR2, or the BMPR2-E376K variant. As expected, SMAD1/5/9 phosphorylation and its downstream target ID1 were detected only in the cells expressing BMPR2-E376K (see FIG. 8). Consistently, cells expressing BMPR2-E376K were positive for ALP staining (see FIG. 3f). Addition of BMP2 or BMP4 increased the ALP expression in both BMPR2 WT and BMPR2-E376K backgrounds, and it was clear that BMPR2-E376K cells showed higher ALP expression than BMPR2 WT cells (see FIG. 8). In this setting, it was found that treatment with not only dorsomorphin, a potent inhibitor of BMP signaling (see non-patent document 20), but also with 5B431542, a potent inhibitor TGF signaling (see non-patent document 21), reduced ALP staining (see FIGS. 3f to 3g), suggesting that both BMP and TGF signaling activation are important for the heterotopic ossification phenotype of current FOP case.

The present invention reports that a gain-of-function mutation in the BMPR2 gene is causative for an inherited skeletal dysplasia, FOP, characterized by heterotopic bone formation in places where soft tissue should grow. To date, ACVR1 is the only known gene responsible for FOP, and more than 95% of FOP patients harbor the specific R206H mutation in the ACVR1 gene (see non-patent documents 6 and 7). However, due to the limited genetic causes identified in FOP patients, it is not yet clear how ACVR1-R206H induces constitutive activation of BMP signals and the pathophysiology of FOP in general. In the present invention, it was described that an individual presented with typical FOP phenotypes, except for the big toe anomaly. From the genetic sequencing analysis, it was found that the individual did not have a mutation in ACVR1, but instead harbored a novel gain-of-function mutation in the BMPR2 gene. The pathogenicity of the BMPR2-E376K mutation was validated with multiple functional assays, and the inventors found several interesting aspects of the BMPR2-E376K mutation, which will be informative to understand not only the pathogenesis of FOP, but the nature of TGF/BMP signaling cascades as well.

The inventors found that BMPR2-E376K variant is consistently associated with the type I receptor ACVR1, which increases the proximity between type I and type II TGF-beta receptors even in the absence of BMP ligands (see FIG. 3a). The molecular consequences of the ACVR1-R206H mutant remain elusive. Interestingly, it was found that combined expression of ACVR1-R206H and BMPR2-E376K showed additive effects (see FIG. 3b) on inducing BMP signals, implying that ACVR1-R206H might have different molecular mechanisms of activation of BMP signaling cascades. Nonetheless, the inventors believe that these findings will be not only informative to understand the exact pathophysiology of FOP arising from the ACVR1-R206H mutation, but also useful to develop drugs that inhibit enhanced BMP signaling, which will be applicable to FOP and other diseases as well.

It was reported that treatment with activin A in the presence of the ACVR1-R206H mutant further stimulates SMAD1/5/9 phosphorylation (see non-patent documents 11 and 12). To date, there is no explanation for the activation of BMP signals in the ACVR1-R206H background, although activin A is normally known to induce TGF-beta signaling. The inventors demonstrated that the BMPR2-E376K mutant and the ACVR1-R206H mutant have a different molecular basis for inducing BMP signals. However, similar to ACVR1-R206H, it was found that BMPR2-E376K is also able to further induce BMP signals upon activin A treatment (see FIG. 3c). These findings suggest that the molecular mechanisms of enhanced BMP signaling in response to activin A in FOP could be more general rather than depending on specific molecular backgrounds. Our findings will be informative for understanding the functional roles of activin A in FOP, although further studies will be required.

Loss-of-function mutations of BMPR2 have been well described in pulmonary artery hypertension (PAH) (see non-patent documents 22 and 23). It was proposed that loss of BMPR2 function results in enhanced TGF-beta signaling cascades, leading to hyperproliferation of smooth muscle cells in blood vessels, although the exact molecular basis of PAH remains elusive (see non-patent document 24). Here the inventors report the first gain-of-function BMPR2 mutation and its potentially causative role in human disease. These findings clearly demonstrate that the constitutive activation of BMPR2 enhances BMP signaling, which results in heterotopic bone formation phenotype. Understanding of the physiological functions of BMPR2 will also contribute to our understanding of the pathophysiology of PAH, and set the stage for developing new treatment options.

Unlike the ACVR1-R206H mutant, cells expressing BMPR2-E376K showed SMAD2 phosphorylation and downstream target gene expression. In further studies, it was identified that the type I receptor responsible for SMAD2 phosphorylation is TGFBR1, suggesting that in some cases there might be crosstalk between BMP and TGF signaling. It was also found that the activated SMAD2-dependent signaling is partly involved in the processes of heterotopic ossification, which is supported by other studies showing that TGF-beta signaling is critical for FOP phenotypes (see non-patent document 25). It will be worth trying novel therapeutic approaches with FOP that focus on inhibiting TGF-beta signaling.

Although the exemplary embodiments of the present invention have been described in order to achieve the technical objectives, it is understood that various changes and modifications can be made by one with ordinary skilled in the art within the spirit and scope of the present invention as hereinafter claimed.

1. A BMPR2-E376K mutant in which an amino acid 376 of a bone morphogenetic protein type 2 receptor (BMPR2) gene encoding BMPR2 is mutated from glutamic acid (E) to lysine (K).

2. The BMPR2-E376K mutant of claim 1, wherein the mutant is characterized by having a point mutation of guanine (G) to adenine (A) at nucleotide 1126.

3. The BMPR2-E376K mutant of claim 1, wherein the mutant is characterized by causing a phenotype of Fibrodysplasia ossificans progressiva (FOP).

4. The BMPR2-E376K mutant of claim 1, wherein the mutant is characterized by being used to treat bone disease through osteogenic differentiation.

5. A cell line including the mutant of claim 1.


SEQUENCE LISTING (55 sequences)

SEQ ID NO: 1 - DNA, 53 bp, Artificial Sequence
  BMPR2-F Primer
ggggacaagtttgtacaaaaaagcaggcttaatgacttcctcgctgcagcggc

SEQ ID NO: 2 - DNA, 48 bp, Artificial Sequence
  BMPR2-R Primer
ggggaccactttgtacaagaaagctgggtctcacagacagttcattcc

SEQ ID NO: 3 - DNA, 23 bp, Artificial Sequence
  Target sequence for CRISPR-Cas9
agataatgcagccataagcaagg

SEQ ID NO: 4 - DNA, 120 bp, Artificial Sequence
  donor template
ccatgaggctgactggaaatagactggtgcgcccaggggaggaagataatgcagccatctccgaggtgagtgtatacaaaaggtatcacactgatgtactttgaaatgataatttaatta

SEQ ID NO: 5 - DNA, 20 bp, Artificial Sequence
  hBMPR2 E7 F1
gcctccttttacagccctat

SEQ ID NO: 6 - DNA, 20 bp, Artificial Sequence
  hBMPR2 E7 R1
aactttacccttgcctcaaa

SEQ ID NO: 7 - DNA, 51 bp, Artificial Sequence
  hBMPR2 E7 dF1
acactctttccctacacgacgctcttccgatctacagcagaaatgtcctag

SEQ ID NO: 8 - DNA, 52 bp, Artificial Sequence
  hBMPR2 E7 dR1
gtgactggagttcagacgtgtgctcttccgatctctctttaccttaggtgat

SEQ ID NO: 9 - DNA, 21 bp, Artificial Sequence
  mCol2a1 sense primer
cctccgtctactgtccactga

SEQ ID NO: 10 - DNA, 20 bp, Artificial Sequence
  mCol2a1 antisense primer
attggagccctggatgagca

SEQ ID NO: 11 - DNA, 20 bp, Artificial Sequence
  mCol0a1 sense primer
aacaggtatgcccgtgtctg

SEQ ID NO: 12 - DNA, 20 bp, Artificial Sequence
  mCol0a1 antisense primer
tcatcaaatgggatgggggc

SEQ ID NO: 13 - DNA, 20 bp, Artificial Sequence
  mAggrecan sense primer
tggcttctggagacaggact

SEQ ID NO: 14 - DNA, 20 bp, Artificial Sequence
  mAggrecan antisense primer
ttctgctgtctgggtctcct

SEQ ID NO: 15 - DNA, 23 bp, Artificial Sequence
  mGapdh sense primer
catgttccagtatgactccactc

SEQ ID NO: 16 - DNA, 20 bp, Artificial Sequence
  mGapdh antisense primer
ggcctcaccccatttgatgt

SEQ ID NO: 17 - DNA, 19 bp, Artificial Sequence
  hPAl-1 sense primer
tcctggttctgcccaagtt

SEQ ID NO: 18 - DNA, 20 bp, Artificial Sequence
  hPAl-1 antisense primer
ccaggttctctaggggcttc

SEQ ID NO: 19 - DNA, 20 bp, Artificial Sequence
  hPDGFB sense primer
ctggcatgcaagtgtgagac

SEQ ID NO: 20 - DNA, 19 bp, Artificial Sequence
  hPDGFB antisense primer
cgaatggtcacccgagttt

SEQ ID NO: 21 - DNA, 20 bp, Artificial Sequence
  hTHBS-1 sense primer
caatgccacagttcctgatg

SEQ ID NO: 22 - DNA, 18 bp, Artificial Sequence
  hTHBS-1 antisense primer
tggagaccagccatcgtc

SEQ ID NO: 23 - DNA, 19 bp, Artificial Sequence
  hGAPDH sense primer
agccacatcgctcagacac

SEQ ID NO: 24 - DNA, 19 bp, Artificial Sequence
  hGAPDH antisense primer
gcccaatacgaccaaatcc

SEQ ID NO: 25 - DNA, 17 bp, Artificial Sequence
  FIG. 1c_Father
gccataagcgaggtgag

SEQ ID NO: 26 - DNA, 17 bp, Artificial Sequence
  FIG. 1c_Mother
gccataagcgaggtgag

SEQ ID NO: 27 - DNA, 17 bp, Artificial Sequence
  FIG. 1c_Sibling
gccataagcgaggtgag

SEQ ID NO: 28 - DNA, 17 bp, Artificial Sequence
  FIG. 1c_Proband_c.1126G>A
gccataagcraggtgag

SEQ ID NO: 29 - protein, 20 aa, Artificial Sequence
  FIG. 1e_BMPR2_Patient
NRLVRPGEEDNAAISKVGTI

SEQ ID NO: 30 - protein, 20 aa, Artificial Sequence
  FIG. 1e_BMPR2_Homo sapiens
NRLVRPGEEDNAAISEVGTI

SEQ ID NO: 31 - protein, 20 aa, Artificial Sequence
  FIG. 1e_BMPR2_Mus musculus
NRLVRPGEEDNAAISEVGTI

SEQ ID NO: 32 - protein, 20 aa, Artificial Sequence
  FIG. 1e_BMPR2_Rattus norvegicus
NRLVRPGEEDNAAISEVGTI

SEQ ID NO: 33 - protein, 20 aa, Artificial Sequence
  FIG. 1e_BMPR2_Bos taurus
NRLVRPGEEDNAAISEVGTI

SEQ ID NO: 34 - protein, 20 aa, Artificial Sequence
  FIG. 1e_BMPR2_Gallus gallus
NRLVRPGEEDNAAISEVGTI

SEQ ID NO: 35 - protein, 20 aa, Artificial Sequence
  FIG. 1e_BMPR2_Callithrix jacchus
NRLVRPGEEDNAAISEVGTI

SEQ ID NO: 36 - protein, 20 aa, Artificial Sequence
  FIG. 1e_BMPR2_Sus scrofa
NRLVRPGEEDNAAISEVGTI

SEQ ID NO: 37 - DNA, 24 bp, Artificial Sequence
  FIG. 2a_mutant
gcagccataagcaaggtgagtgta

SEQ ID NO: 38 - DNA, 24 bp, Artificial Sequence
  FIG. 2a_WT
gcagccataagcgaggtgagtgta

SEQ ID NO: 39 - DNA, 24 bp, Artificial Sequence
  FIG. 2a_WT
gcagccataagcgaggtgagtgta

SEQ ID NO: 40 - DNA, 24 bp, Artificial Sequence
  FIG. 2a_KO #3
  misc_difference (9)..(9): replace= “-” for a deletion
gcagccataagcaaggtgagtgta

SEQ ID NO: 41 - DNA, 24 bp, Artificial Sequence
  FIG. 2a_WT
gcagccataagcgaggtgagtgta

SEQ ID NO: 42 - DNA, 25 bp, Artificial Sequence
  FIG. 2a_KO #5
gcagccataaagcaaggtgagtgta

SEQ ID NO: 43 - DNA, 24 bp, Artificial Sequence
  FIG. 2a_WT
gcagccataagcgaggtgagtgta

SEQ ID NO: 44 - DNA, 24 bp, Artificial Sequence
  FIG. 2a_KI #203
gcagccatctccgaggtgagtgta

SEQ ID NO: 45 - protein, 5 aa, Artificial Sequence
  FIG. 2a_mutant_PRT
AAISK

SEQ ID NO: 46 - protein, 5 aa, Artificial Sequence
  FIG. 2a_WT_PRT
AAISE

SEQ ID NO: 47 - protein, 5 aa, Artificial Sequence
  FIG. 2a_KO #3_PRT
AAIAR

SEQ ID NO: 48 - protein, 5 aa, Artificial Sequence
  FIG. 2a_KO #5_PRT
AAIKQ

SEQ ID NO: 49 - protein, 5 aa, Artificial Sequence
  FIG. 2a_KI #203_PRT
AAISE

SEQ ID NO: 50 - DNA, 35 bp, Artificial Sequence
  FIG. 4a_mutant allele
ggaagataatgcagccataagcaaggtgagtgtat

SEQ ID NO: 51 - DNA, 35 bp, Artificial Sequence
  FIG. 4a_Donor template
ggaagataatgcagccatctccgaggtgagtgtat

SEQ ID NO: 52 - DNA, 35 bp, Artificial Sequence; identical to SEQ ID NO: 51
  FIG. 4a_Corrected template

SEQ ID NO: 53 - DNA, 35 bp, Artificial Sequence; identical to SEQ ID NO: 50
  FIG. 4b_mutant allele

SEQ ID NO: 54 - DNA, 35 bp, Artificial Sequence
  FIG. 4b_Insertion
gaagataatgcagccataaagcaaggtgagtgtat

SEQ ID NO: 55 - DNA, 35 bp, Artificial Sequence; identical to SEQ ID NO: 50
  FIG. 4b_Deletion
  misc_difference (19)..(19): replace= “-” for a deletion